(19) United States
(12) Patent Application Publication
(10) Pub. No.: US 2013/0111611 A1
(43) Pub. Date: May 2, 2013

(54) METHOD TO MEASURE THE METABOLIC RATE OR RATE OF GLUCOSE CONSUMPTION OF CELLS OR TISSUES WITH HIGH SPATIOTEMPORAL RESOLUTION USING A GLUCOSE NANOSENSOR

(71) Applicants: Luis Felipe Barros Almedo, Valdivia (CL); Carla Ximena Bittner Hofmann, Valdivia (CL)

(72) Inventors: Luis Felipe Barros Almedo, Valdivia (CL); Carla Ximena Bittner Hofmann, Valdivia (CL)

(73) Assignees: CARNEGIE, INSTITUTION OF WASHINGTON, Washington, DC (US); CENTRO DE ESTUDIOS CIENTIFICOS DE VALDIVIA, Valdivia (CL)

(21) Appl. No.: 13/807,150

(22) PCT Filed: Jun. 30, 2010
(86) PCT No.: PCT/US10/40643

§ 371 Date: Dec. 27, 2012

(51) Int. Cl. C12Q 1/54; C12Q 1/02

(52) U.S. Cl. CPC C12Q 1/54; C12Q 1/025
USPC 800/3; 435/14; 506/10

(57) ABSTRACT

A method to measure the metabolic rate or rate of glucose consumption of cells or tissues with high spatiotemporal resolution using a glucose nanosensor is disclosed. The method generally includes providing single cells or cell populations, cells in suspension or adherent, in a cell culture, a tissue culture, a mixed cell culture, a tissue explant, or in animal tissues in vivo for the measurement of glucose metabolic rate; expressing a glucose sensor in individual cells; calibrating the sensor in controlled conditions; disrupting the steady-state of glucose entering the cell; recording the output from the sensor and calculating the corresponding glucose concentration at different times; and determining the glucose metabolic rate. The method can be applied for the screening of molecules with pharmacological potential, determination of glucose rate of cancerous cells, tissue physiology and biochemistry research.

### FIELD OF THE INVENTION

The present invention is a new method to measure the metabolic rate or rate of glucose consumption of cells or tissues with high spatiotemporal resolution using a glucose nanosensor. This method can be applied for the screening of molecules with pharmacological potential, determination of glucose rate of cancerous cells, tissue physiology and biochemistry research.

### BACKGROUND OF THE INVENTION

The metabolic rate is the speed at which the body burns fuel and is sensitive to physiological activity, hormones, stress, aging and malignant transformation. Inside tissues, every cell is characterized by a specific metabolic rate, ranging from low for quiescent cells like skin fibroblasts to very high for some cells in epithelia and muscle. The metabolic rate of an individual cell can also vary through time: for instance, adipocytes increase their rate of glucose uptake by up to ten-fold in response to insulin, whereas neurons may raise their energy demand by larger factors in response to electrical stimulation. The metabolic rate is also affected by aging and disease. For example cancer cells show higher metabolic rates than their surrounding tissue, a phenomenon involved in tumor progression and instrumental for the purposes of diagnosis, staging and prognosis of this disease^{1,2}.

There are no available methods to measure the metabolic rate in single cells. More specifically, current and common techniques to measure the metabolic rate using radioactive isotopes can not resolve single cells and have poor temporal resolution, which hampers the study of complex tissues or fast phenomena.

Cell populations and tissues are usually studied by measuring the uptake of radioactive metabolites. In a typical in vitro experiment, a million cells in a culture dish are exposed for 20 minutes to radiolabeled deoxyglucose, which is phosphorylated by hexokinase, so that the radioactivity trapped inside the cells is assumed to be proportional to the metabolic rate. For in vivo experiments, the tracer is given intravenously, and after a period of 20 minutes or more, the radioactivity accumulated in the tissue is detected by autoradiography or non-invasively by Fluorodeoxyglucose (FDG)-PET scanning, techniques that have found wide application in functional mapping of the brain and for the detection of tumors. Useful as they are, deoxyglucose uptake, deoxyglucose autoradiography and FDG-PET scanning have their limitations. Firstly, they do not use glucose but an analog, which is not handled by the cell in the same way. Secondly, uptake of glucose analogs is not just determined by metabolic rate but also depends on the usually unknown properties of the glucose transporters that mediate their entry into the cell. Thirdly, they offer low spatiotemporal resolution, which precludes resolving the contribution of individual cells or detecting rapid phenomena; and finally, they are relatively insensitive and require isotope manipulation, which makes them inadequate for the purposes of high-throughput analysis.

Furthermore, as presently known, all patent applications related to metabolic rate determination are directed to the measurement of glucose, but no patent application has addressed the issue of metabolic rate determination. In particular, US patent application 20050197311 where compositions and methods for measuring intracellular glucose are described. This application uses nucleic acid constructs comprising a glucose-regulated mRNA instability element. This instability element is coupled to a reporter vector which will be downregulated in the presence of high glucose concentrations. The limitation of this method is the delay in protein expression and therefore, the method is not suitable for applications where a good time resolution is required, as in the case of determining the metabolic rate of single cells or tissues. The present invention allows temporal resolution of minutes and even seconds compared to the temporal resolution of hours achieved by the description found in US patent application 20050197311.

US patent application 20050118726 describes a sensor comprising different protein domains based on a fusion protein comprising fluorescent domains. It also describes potential devices for the detection of glucose concentration. Nevertheless, it does refer to the temporal resolution of the sensor and it does not address the issue of metabolic rate determination.

Fluorescence allows estimation of metabolite concentration with high sensitivity and spatiotemporal resolution. In addition to NAD(P)H autofluorescence³, the increasing availability of DNA-encoded probes has made it possible to measure the concentration of several metabolites using fluorescence^{4,5}. Whereas metabolite concentrations are interesting per se, the presence of homeostatic mechanisms in living cells makes steady-state concentrations of little value for the purposes of flux prediction.

It is possible nowadays to measure the concentration of glucose in single cells using FRET nanosensors^{4,6,7}.

International patent application WO2006006166 describes a glucose sensor based on yellow fluorescent protein (YFP) and cyan fluorescente protein (CFP), U.S. Pat. No. 7,432,353 describes a maltose sensor comprising different moieties based on fluorescent proteins such as enhanced yellow, cyan, blue and green fluorescent proteins and red fluorescent protein. International patent application WO2007046786 describes a glucose fluorescent sensor. Although all these patent applications mention a fluorescent glucose or maltose sensor, none of them addresses the issue of determination of the metabolic rate in single cells or tissues.

However, as with any other metabolite, the concentration of glucose in the steady-state is not informative about flux through the metabolic pathway. The present invention provides a method to approach glucose flux.

In the present invention, the inconvenient of measuring glucose concentration in steady-state has been circumvented by interrupting the steady-state, while measuring the concentration of glucose with a DNA-encoded nanosensor. In particular, the method of the present invention allowed for the first time to observe that astrocytic glycolysis can be activated by neuronal signals within seconds, supporting central roles for astrocytes in neurometabolic and neurovascular coupling in the brain. It was also possible to make a direct comparison of metabolism in neurons and astrocytes lying in close proximity, opening the way to a high resolution characterization of brain energy metabolism. Single-cell metabolic rates have also been measured in fibroblasts, adipocytes, myoblasts and tumor cells, evidencing metabolic heterogeneity, even for cell lines. The method of the present invention allows the investigation of tissue metabolism at the single cell level and is readily adaptable for high-throughput analysis using microtiter plates.

### BRIEF DESCRIPTION OF THE INVENTION

The present invention addresses the issues related to glucose measurements to determine the metabolic rate arising from the limitations imposed by steady-state, by disrupting the steady-state. The disruption of the steady-state to perform analyte measurements is not obvious considering the previous art, since all the usual and current methods rely on the assumption of steady-state, and therefore, providing the conditions to keep the steady-state, instead of disrupting it. The disruption of steady-state will only provide useful results and information if the method, analyte sensor, and proper interpretation of data are achieved.

The invention comprises a method for the measurement of glucose metabolic rate. The method can be applied to single cells or cell populations, cells in suspension or adherent, to a cell culture, a tissue culture, a mixed cell culture, a tissue explant, or it can also be applied to animal tissues in vivo. The method comprises the expression of a suitable glucose sensor in individual cells. The glucose sensor should be able to monitor the glucose concentration in real-time. Suitable glucose sensors should provide an easy to read signal in a glucose concentration-dependent manner and be insensitive to other molecules commonly present in cells. The expression, presence or degradation of the sensor should not interfere significantly with the cell metabolism. A non limiting example of such a sensor would be the one described in patent application WO2007046786.

The glucose sensor should be expressed in single cells or cell populations, cells in suspension or adherent, in a cell culture, a tissue culture, a mixed cell culture, a tissue explant, or in animal tissues in vivo. When using a genetically-encoded glucose sensor, the gene expression can be attained by any suitable method to transfer the sensor gene information to the host cell. Examples of gene transfer methodologies are plasmid transfer for instance using liposomal delivery, virus transfer and transgenesis.

Once the sensor is expressed in single cells or cell populations, cells in suspension or adherent, in a cell culture, a tissue culture, a mixed cell culture, a tissue explant, or in animal tissues in vivo, the sensor is calibrated according to pre-established conditions.

With the information obtained in the calibration step, the determination of the metabolic rate is carried out by disrupting the flux of glucose, which is normally maintained in a steady-state. The disruption can be attained by any suitable method. In one embodiment, the disruption of the steady-state is obtained by changing the extracellular concentration of glucose to a level which is inferior to the intracellular glucose level. In another embodiment, the disruption of the steady-state is attained by adding an inhibitor of the glucose transporter GLUT, such as, but not limited to cytochalasin B, phloretin, genistein, parachloromercurybenzoate, anti-GLUT antisera, etc. In cells were glucose transport is mediated by Na₊-dependent transporters, the method can be applied using phloridzin.

Once the disruption of the steady-state is obtained, the registry of data produced by the sensor allows the determination of the metabolic rate.

Summarizing, the method of the present invention comprises the following steps:
Providing a system for the measurement of glucose metabolic rate. The system can be single cells or cell populations, cells in suspension or adherent, a cell culture, a tissue culture, a mixed cell culture, a tissue explant, or animal tissues in vivo;
Expressing a suitable glucose sensor in individual cells. The gene expression single cells or cell populations, cells in suspension or adherent, in a cell culture, a tissue culture, a mixed cell culture, a tissue explant, or in animal tissues in vivo;
Calibrating the sensor in the controlled conditions;
Disrupting the steady-state of glucose entering the cell by:
   Exposing the extracellular space to a variation in the concentration of glucose (ETM: Equilibrium Transport Method) or
   Adding a GLUT inhibitor (ITM; Inhibitor Transport Method);
Recording the output from the sensor and calculate the corresponding glucose concentration at different times; and
Determining the glucose metabolic rate.

The preferred type of cells or tissue are those that express equilibrative glucose transporters and do not express Na⁺-dependent glucose transporters, which include neurons, astrocytes, muscle cells, adipocytes, liver cells, pancreatic cells, fibroblasts, stem cells, blood cells, endothelial cells, and most mammalian cells types. For cells that express the Na⁺-dependent glucose transporters, such as intestinal cells and kidney cells, the metabolic rate can be measured by interrupting the flux of glucose using the inhibitor phloridzin.

### BRIEF DESCRIPTION OF THE DRAWINGS

The invention is illustrated by the accompanying drawings wherein:

FIGS. 1(a)-(c) schematically illustrate the two variants of the method identified as ETM and ITM of the invention.

FIGS. 2(a)-(c) demonstrate ETM variant of the method used in astrocytes.

FIGS. 3(a)-(f) demonstrate ITM variant of the method in astrocytes.

FIGS. 4(a) and 4(b) illustrate temporal resolution involving activation of astrocytic glycolysis by neuronal signals using ITM.

FIGS. 5(a)-(d) illustrate spatial resolution involving simultaneous measurement of glycolytic rate in astrocytes and neurons.

FIGS. 6 (a)-(f) comprise graphs showing the heterogeneity of glycolytic rates in various cells determined by using ITM.

FIG. 7 (a)-(d) Calibration of the FLII12 Pglu600Δμ6 nanosensor. The nanosensor was calibrated in astrocytes (a), 3T3-L1 fibroblasts (b), C2C21 myoblasts (c) and HeLa cells (d) by exposing the cultures to increasing concentrations of glucose (0-2 mM) after full inhibition of glycolysis by preincubation with iodoacetic acid for 30 min. Data are from at least 7 cells from three experiments for each cell type. The saturation parameters were obtained by fitting a rectangular hyperbola to the data using non-linear regression.

FIG. 8 (a)-(c) Effect of glutamate/K⁺ on astrocytic cell volume. a) A calcein-loaded cell was first exposed to 50 μM glutamate/15 mM K⁺ and then to a solution in which NaCl had been reduced to make the solution 30% hypotonic (hypo). Relative calcein concentration was calculated from calcein fluorescence using the response to hypotonicity as a calibration factor. b) The initial time course of the response to glutamate/K⁺ shown in A (calcein) is plotted together with an example of relative decrease in glucose concentration elicited by K⁺ (glucose, same data as shown in FIG. 4a). c) Initial rates of decrease are given for a series of experiments with 50 μM glutamate/15 mM K⁺ for glucose (n=25 cells in five experiments) and calcein (n=20 cells in three experiments).

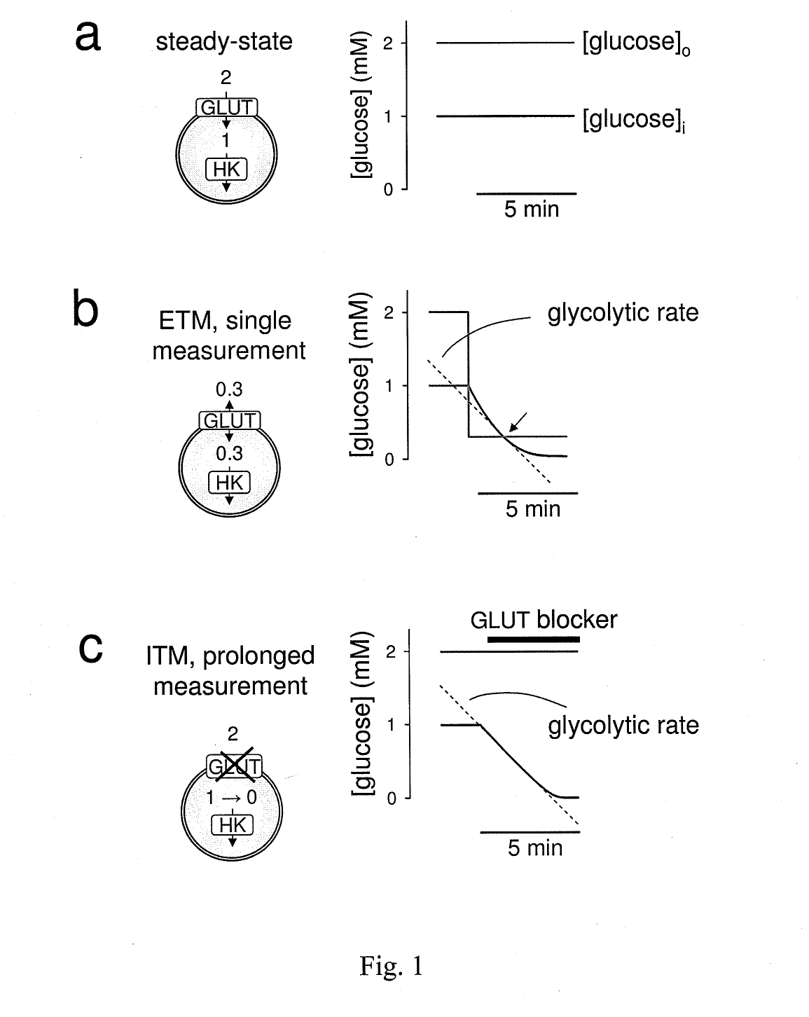

### DETAILED DESCRIPTION OF THE INVENTION

As mentioned before, the method of the present invention is directed to the measurement of the metabolic rate in different systems. In particular, the method of the present invention is based in the disruption of the glucose steady-state.

The disruption of the glucose steady-state can be achieved by different means. In one embodiment, the disruption of the glucose steady-state can be achieved by reducing extracellular glucose in a single step to a concentration lower than intracellular glucose but different from zero. Such perturbation leads to a rapid decrease towards a new steady-state. Critically, there is an instant in the course of the decay when glucose inside and outside are identical. Because the transporters that mediate glucose uptake into most mammalian cells are equilibrative, that is, they only move glucose when there is a concentration gradient across the plasma membrane, in the moment at which glucose is equilibrated, there will be no net flux through the transporter, or for that matter through simple diffusion or any other passive pathway. Thus eliminated the contribution of permeability, the rate of glucose concentration decrease at glucose equilibrium will be identical to the rate of hexokinase, i.e. the rate of glycolysis at its entry point.

In another embodiment of the invention the disruption of the glucose steady-state is achieved by eliminating the contribution of the permeability by pharmacological block of the glucose transporter. The inhibition of the transporter causes a quasi-linear fall in intracellular glucose, a linearity that results from the high affinity of hexokinase for glucose, which remains nearly saturated as glucose falls from the millimolar range into the hundreds of micromolar. Thus the rate of glucose decrease after blocking transport also corresponds to the glycolytic rate and is a good estimate of the Vmax of hexokinase.

The method of the present invention can be better explained in 6 steps:

First step: Providing a system for the measurement of glucose metabolic rate.

The system can be cells in isolation, a cell culture, a tissue culture, a mixed cell culture, in tissue explant or a tissue in a living animal. In non-limiting illustrative examples, the system can be a cell culture of astrocytes, neurons, fibroblasts, adipocytes or muscle cells.

Second step: Expressing a suitable glucose sensor in individual cells.

The glucose sensor should be able to monitor the glucose concentration in real-time. Suitable glucose sensors should provide an easy to read signal in a glucose concentration-dependent manner and be insensitive to other molecules commonly present in cells. The expression, presence or degradation of the sensor should not interfere significantly with the cell metabolism. In a non-limiting illustrative example, the glucose sensor can be FLIPglu600μΔ11, described in Patent application WO2007046786.

If the sensor is genetically-encoded, the gene expression can be attained by any genetic-engineering method, for instance plasmid transfer, virus transfer or transgenesis.

Third step: Calibrating the sensor in controlled conditions.

The sensor is calibrated firstly by pre-treating the cells for 30 minutes with 0.5 mM iodoacetic acid to block glycolysis. In the absence of glycolytic flux, the presence of equilibrative GLUT transporters makes the concentration of glucose inside and outside the cell identical. Next, the cells are exposed to increasing concentrations of glucose and the signal given by the sensor is monitored. Sensor signal is plotted versus concentration to estimate the saturation parameters. This procedure is carried out once for each different cell type. For subsequent measurements, the signal is measured transiently in cells in the absence of glucose. This “zero” reading is used together with the aforementioned saturation parameters to transform sensor signal into glucose concentration.

Fourth step: Disrupting the steady-state of glucose entering the cell.

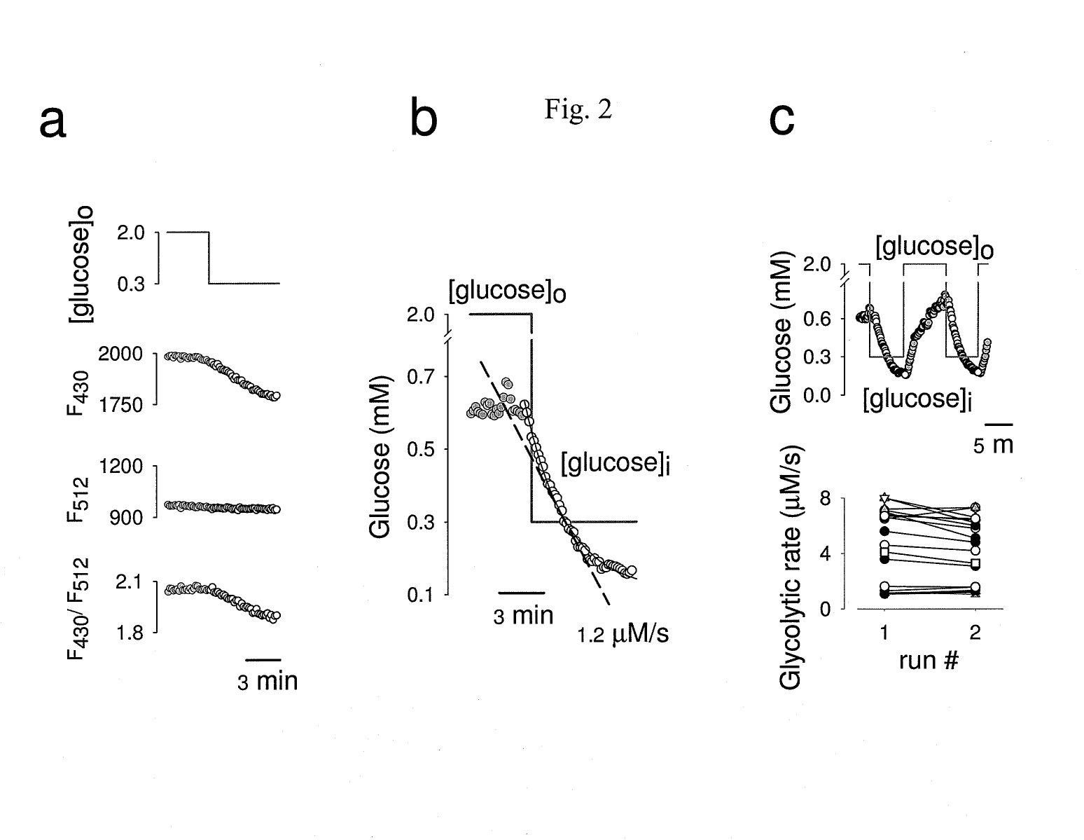

The disruption of the steady-state of glucose can be obtained by exposing the extracellular space to a variation in the concentration of glucose (ETM). In a non-limiting illustrative example, the extracellular glucose concentration is lowered from 2 mM to 0.3 mM. This change in extracellular glucose causes a progressive decline in intracellular glucose concentration, from 1 mM to 0.1 mM (FIG. 2b). The assay has been repeated several times, and as illustrated in FIG. 2c, the rate was not affected by a previous measurement, suggesting that the assay itself does not perturb glycolysis. This insensitivity of metabolism to a moderate decrease in intracellular glucose is consistent with the constancy of metabolic flux while hexokinase remains saturated.

In another embodiment, the disruption of the glucose steady-state can be attained by blocking the glucose transporter GLUT with a suitable inhibitor (ITM). Non-limiting illustrative examples of such inhibitors are cytochalasin B, phloretin, genistein, parachloromercurybenzoate and anti-GLUT antisera. The inhibitor is added to the culture medium in a concentration that blocks at least 90% of the transporter, for cytochalasin B the range is 5 μM to 20 μM.

Fifth step: Recording the output from the sensor and calculate the corresponding glucose concentration at different times.

Independent of the embodiment of the method used to disrupt the steady-state, the reading from the sensor is registered in time, considering appropriate intervals of time, ranging from 100 ms to 1 minute, for example, every 100 ms, every second, every 10 s, every 1 minute. The output from the sensor reading and the calibration curve allows the calculation of glucose concentration at each time point.

Sixth step: Determining the glucose metabolic rate.

Independent of the embodiment of the method used to disrupt the steady-state, the rate of glycolysis is estimated by fitting a monoexponential function to the time course of decay. In one embodiment, the metabolic rate is computed from the rate of glucose concentration decrease at the point when the intracellular concentration becomes equal to the extracellular concentration, for instance at 0.3 mM (interrupted line in FIG. 2b). In a second embodiment, in the presence of a GLUT blocker, the metabolic rate is computed from the rate of glucose concentration decrease at any glucose concentration.

In a further embodiment, the method can be incorporated in a diagnostic kit for the purposes of metabolic rate measurement in human or animal tissue samples.

The method of the invention can be applied to determine the metabolic rate in any situation it is needed.

Cancer cells are characterized by a substantial increase in their rate of glycolysis, so called a glycolytic phenotype, which is important for their capacity to form metastasis. Pharmacological reversion of the glycolytic phenotype has been shown to cause partial reversal of the cancerous phenotype. In one embodiment, the method can be applied to determine the metabolic rate in a cancer biopsy, thus helping to evaluate the rate of glycolysis in the cells and therefore how aggressive is the cancer and the most appropriate course of action and drugs to be administered. Cancer types that can be subjected to analysis according to the method of the present invention are selected, but not limited to breast cancer, bladder cancer, colon cancer, glioblastoma, lung cancer, hepatocellular carcinoma, gastric cancer, melanoma, thyroid cancer, endometrial cancer, kidney cancer, cervix cancer, pancreatic cancer, esophagus cancer, prostate cancer, brain cancer, ovary cancer, small cell lung cancer, non small cell lung cancer, head and neck cancer, mesothelioma, sarcoma, pediatric malignancies, cholangiocarcinoma.

In other embodiment, the method can be applied in high-throughput manner using a cancer cell line to test the effects of potential anti-cancer drug candidates. Good candidates would be able to decrease the glycolytic rate of the cancer cell line. In particular, the screening of anti-cancer drug candidates would employ the method of the invention in different sets of conditions. Cancerous cell lines suitable for the screening of potential anti-cancer drugs are selected among, but not limited to HeLa cells, Neuro 2A, Caco2, C6, A549, MCF7, PC-3, AGS.

Diabetes mellitus is caused by a decrease in the capacity of muscle cells and adipocytes to metabolize glucose. Hypoglycemic drugs may ameliorate diabetes by increasing the capacity of muscle cells and adipose cells to metabolize glucose. In a further embodiment, the present invention can be applied in a high throughput manner to 3T3-L1 fibroblast and adipocytes, C2C12 myoblasts and myocytes, etc., where a series of potential drug candidates may be tested for its effects in metabolic rate modulation.

In the particular case, where the method can be applied in a high-throughput manner for screening of potential drug candidates, the particular potential drug candidate is added to the culture medium.

### Working Examples

Standard chemicals and tissue culture reagents were from Sigma (St. Louis, Mo.). Constructs coding for the sensors FLIPglu170n, FLIPglu600μΔ11, FLII¹² Pglu600μΔ [symbol] have been described previously^{4,6,7}. Plasmids are available through www.addgene.org. Adenoviral vectors Ad FLIPglu600μΔ11 and Ad FLII¹² Pglu600μ [symbol] 6 were custom made by Vector Biolabs.

### Animals, Cell Culture and Tissue Slices

Animals used were mixed F1 male mice (C57BU6J×CBA/J), kept in an animal room under SPF conditions at a room temperature of 20±2° C., in a 12/12 h light/dark cycle with free access to food and water. All experiments were approved by the Centro de Estudios Científicos Animal Care and Use Committee. Mixed cortical cultures of neuronal and glial cells were prepared from 1-3 day-old neonatal mice as described in Loaiza et al.¹⁹, except that N1-N2/MEM was replaced by B27-supplemented Neurobasal medium (Gibco). Cultures were maintained at 37° C. in a humidified atmosphere of 5% CO₂. At days 5-7, cultures in 35 mm dishes were transfected with 5 μg plasmid DNA using Lipofectamine 2000 (Gibco) or alternatively, exposed to 5×10⁶ PFU of adenoviral vector. Adenoviral vectors showed a very high selectivity for astrocytes over neurons, with a ratio>100. Cell lines were obtained from the ATCC. 3T3-L1 fibroblasts were maintained in Dulbecco's Modified Eagle Medium (DMEM) supplemented with 10% fetal calf serum, 2.5 μg/ml amphotericin B and 100 U/ml penicillin/streptomycin and differentiated into adipocytes as described²⁵ using 5 μg/ml insulin. C2C12 myoblasts and Hela cells were maintained in DMEM supplemented with 10% fetal bovine serum, 2.5 μg/ml amphotericin B and 100 U/ml penicillin/streptomycin. 3T3-L1 fibroblast and adipocytes growing in 10 cm dishes were electroporated with 15 μg plasmid DNA using the BioRad Gene Pulser XCell. HeLa cells in 35 mm dishes were transfected with 5 μg plasmid DNA using Lipofectamine 2000.

Hippocampal slices (200 μm thick) were prepared with a Vibratome 1000 Plus (Warner Instruments) from 15 day-old mice by following standard procedures²⁶. Briefly, brains were immersed in ice-cold aCSF of the following composition (in mM): 125 NaCl, 2.5 KCl, 1.25 NaH₂PO₄, 2.5 MgCl2, 0.5 CaCl₂, 25 glucose, 26 NaHCO₃, that had been previously bubbled with 5% CO₂/95% O₂ for 1 hour to reach pH 7.4. Hippocampi were dissected from coronal slices and incubated for 1-2 hrs in cold dissection medium. The slices were then transferred to 35 mm Petri dishes and cultured at 37° C. in a humidified atmosphere of 5% CO₂ for 5-6 days in 50% (MEM containing 6.5 g/L glucose, 23 mM HEPES and 26 mM NaHCO₃), 25% fetal bovine serum, 25% Hank's solution containing 6.5 g/L glucose, 0.3% glutamine, 1 mM sodium pyruvate, non-essential amino acids, N2 supplement (Invitrogen), 100 U/ml penicillin and 100 μg/ml streptomycin. Slices were infected for 24 hrs with 50×10⁶ PFU of the adenoviral vectors at day 2 of culture. For immunohistochemistry, the slices were fixed overnight in 4% paraformaldehyde in phosphate buffered saline (PBS). After extensive washing with PBS, slices were incubated overnight at 4° C. with rabbit 1:500 anti-cow GFAP antisera (Dako). After further washing with PBS, the tissue was incubated overnight at 4° C. with 1:500 Alexa Fluor 568 goat anti-rabbit IgG (Molecular Probes). Slices were mounted on DakoCytomation medium (Dako) and imaged with a Pascal 5 Zeisss confocal microscope at 488 excitation/505-550 emission (for YFP) and 543 excitation/>580 nm emission (for Alexa Fluor 568).

### Glucose and Volume Measurements

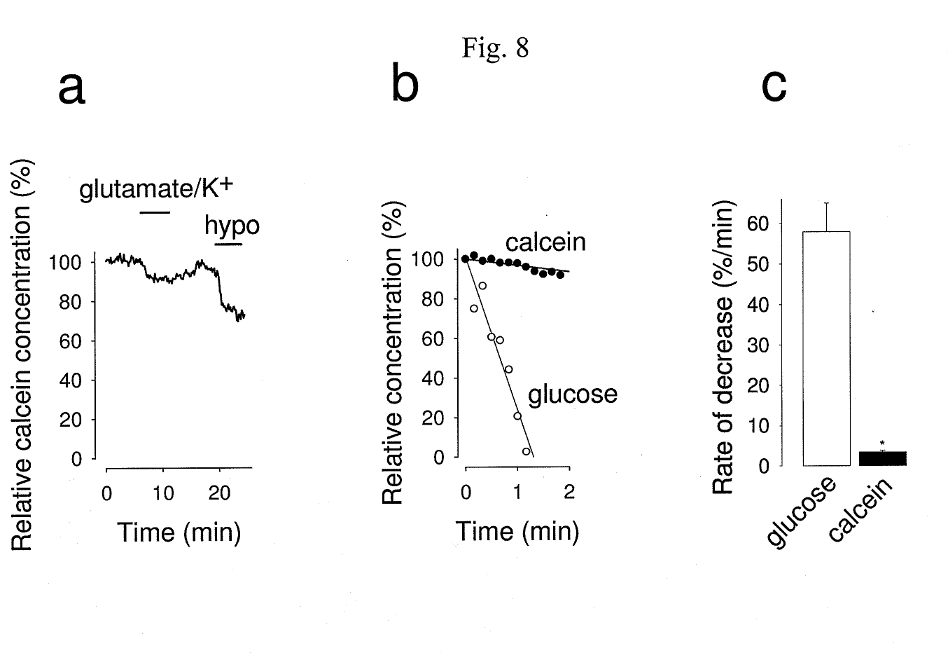

Experiments were carried out at room temperature (22-25° C.). Cultured cells were imaged in HEPES-buffered saline containing (in mM): 136 NaCl, 3 KCl, 1.25 CaCl₂, 1.25 MgSO₄, 1-2 glucose, 2 sodium lactate, 10 HEPES, pH 7.4 or in 95% O2/5% CO₂-gassed buffer of the following composition (in mM): 112 NaCl, 3 KCl, 1.25 CaCl₂, 1.25 MgCl₂, 1-2 glucose, 2 sodium lactate, 10 HEPES, 24 NaHCO₃, pH 7.4. Brain slices were superfused with a 95% O₂/5% CO₂-gassed buffer containing (in mM): 126 NaCl, 3 KCl, 1.25 NaH₂PO₄, 1.25 CaCl₂, 1.25 MgCl₂, 2-3 glucose, 1 sodium lactate, 26 NaHCO₃, pH 7.4. When using higher K⁺ concentrations, NaCl was adjusted to maintain isotonicity. Cultures and slices were imaged with an Olympus IX70 inverted microscope equipped with a 40× oil-immersion objective, a Cairn monochromator with Optosplit (Faversham, UK), and a Hamamatsu Orca camera (Hamamatsu City, Japan) controlled by Kinetics software. For FRET measurements, CFP was excited at 430 nm for 400 ms. Because of the very high FRET efficiency of FLII12 Pglu600μ [symbol] , which results in low CFP signal⁷, we opted for not using the CFP signal in our protocol. Instead, YFP/citrine was excited for 50 ms, which provided a sugar-insensitive denominator for the FRET ratio. The ratio between YFP/Citrine excited by FRET (at 430 nm) and excited directly (at 512 nm) was calibrated by exposing pH-clamped cells to increasing glucose concentrations in the presence of the glycolytic blocker iodoacetic acid. FIG. 8 includes the calibration curves for astrocytes, fibroblasts, myoblasts and HeLa cells. For neurons and adipocytes, whose low glucose permeability made calibration very difficult, we used the calibration curves obtained in astrocytes and fibroblasts, respectively. Relative cell volume was measured using a Pascal 5 Zeisss confocal microscope (optical section <2 μm; 488 nm excitation/505-550 nm emission) in cells ester-loaded with calcein at 0.5 μM for 30 min²⁷. Exposure to anisosmotic solutions showed that over 90% of the calcein fluorescence was volume-sensitive.

### Metabolic Rate Determination in Different Systems

### Determination of Metabolic Rate in Cultured Astrocytes

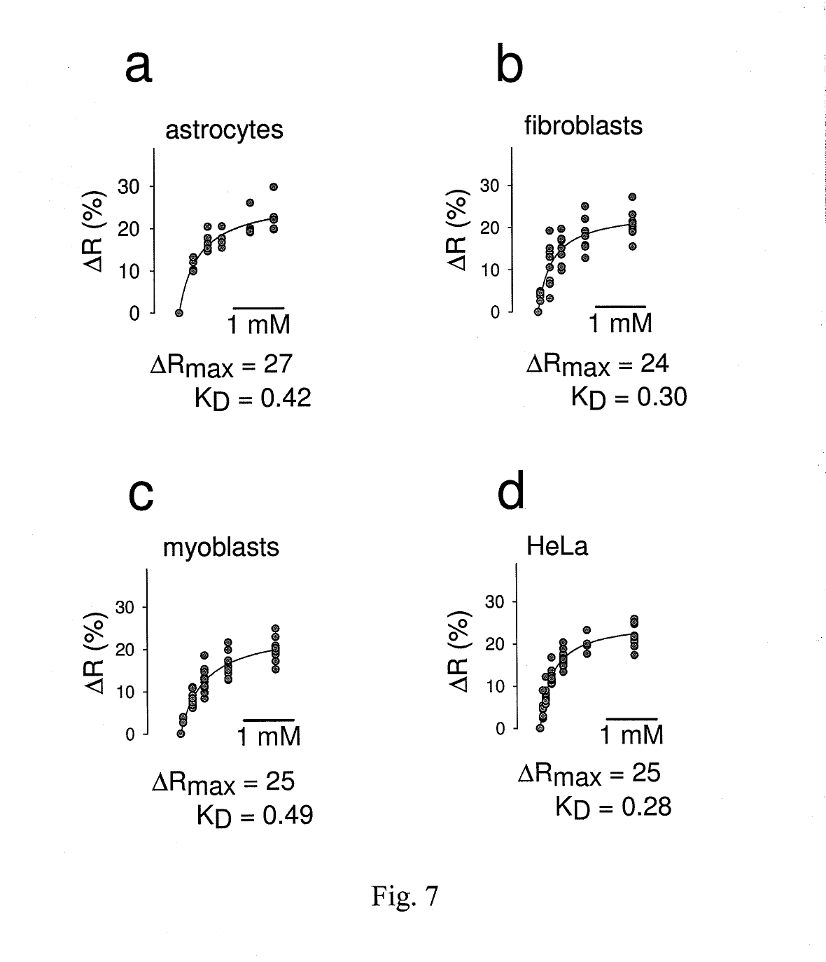

Intracellular glucose was measured in real-time with a DNA-encoded FRET glucose nanosensor^{4,6,7} that was calibrated in situ (FIG. 7). In the presence of 2 mM extracellular glucose, cultured astrocytes maintained a steady-state intracellular glucose concentration averaging 0.63±0.30 mM (n=100 cells in twenty two experiments). The intracellular glucose concentration varied greatly from cell to cell, ranging from 0.2 to 1.9 mM, showing that the balance between glucose permeability and glycolysis is not fixed.

The disruption of the steady-state was obtained by lowering extracellular glucose to 0.3 mM. Lowering extracellular glucose caused a progressive decline in intracellular glucose concentration (FIG. 2b). The rate of glycolysis was estimated by fitting a monoexponential function to the time course of decay and computing the instantaneous slope at 0.3 mM (interrupted line in FIG. 2b) as a metabolic rate of 1.2 μM/s. Next, the assay was repeated several times. As illustrated in FIG. 2c, the rate was not affected by a previous measurement, suggesting that the assay itself does not perturb glycolysis. This insensitivity of metabolism to a moderate decrease in intracellular glucose is consistent with the constancy of metabolic flux while hexokinase remains saturated.

Further to the example above, the method was tested by adding a GLUT inhibitor to the extracellular space.

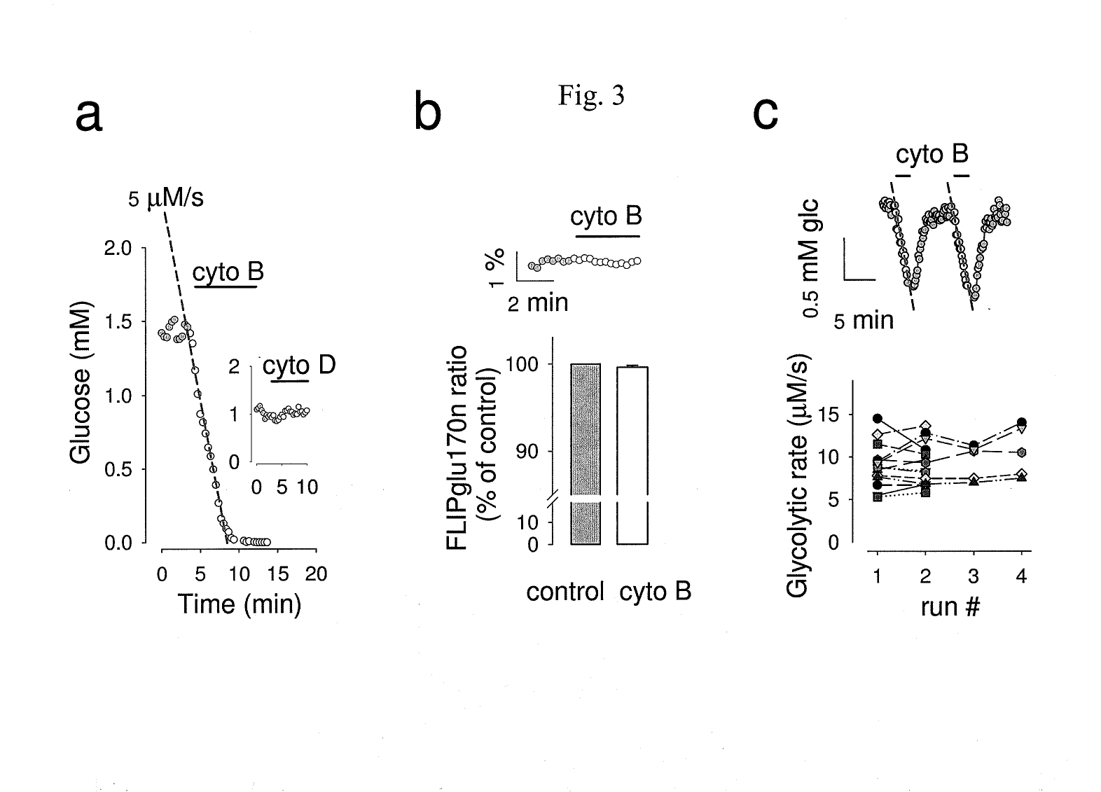

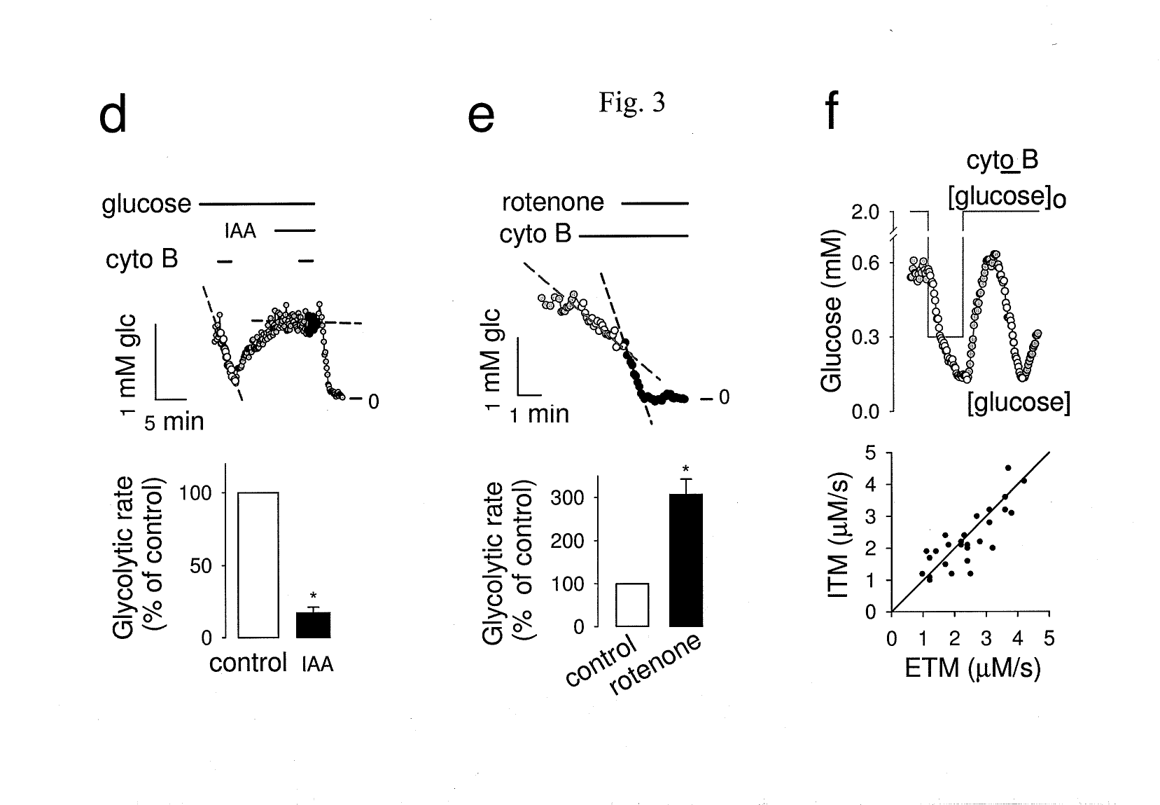

The most widely used inhibitor of the glucose transporter is cytochalasin B, which blocks the transporter isoforms present in most cell types, GLUT1, GLUT3 and GLUT4, with a Kᵢ of about 1 μM or lower¹⁰. Exposure of astrocytes to the inhibitor resulted in a linear decrease in the concentration of glucose (FIG. 3a). In the continuous presence of cytochalasin B, the concentration of glucose reached levels indistinguishable from zero, demonstrating the high degree of transport inhibition achieved. The average rate of glycolysis in cultured astrocytes was 2.0±0.3 μM/s (n=120 cells in thirty experiments) and showed high heterogeneity, with differences of several-fold even inside a microscopic field (data not shown). Several control experiments were designed to check the validity of the disruption of steady-state by adding a GLUT inhibitor. In addition to inhibit the glucose transporter, cytochalasin B affects the actin cytoskeleton, a potential source of interference that can be controlled for with cytochalasin D, a structural analog that targets the cytoskeleton but not the glucose transporter¹⁰. The experiment illustrated in FIG. 3a (inset) supports the specificity of cytochalasin B by showing that cytochalasin D did not affect the concentration of glucose. To control for direct effects of cytochalasin B on the sensor, the inhibitor was applied to galactose-equilibrated cells expressing FLIPglu170n. This very high affinity version of the sensor is saturated in the presence of physiological glucose concentrations⁶, so that any change in its behavior can be unambiguously ascribed to an artifact. As illustrated in FIG. 3b, cytochalasin B did not change the ratio of FLIPglu170n emission intensity, showing that there is no direct effect of the inhibitor on the sensor. Disruption of the steady-state by using a GLUT inhibitor is highly reproducible, confirming that a moderate decrease in intracellular glucose does not affect metabolism (FIG. 3c). Phloretin, a structurally unrelated inhibitor of the glucose transporter, gave a glycolytic rate of 1.7±0.3 μM/s which was not significantly different from the glycolytic rate obtained with cytochalasin B (1.8±0.3 μM/s; n=20 cells in three paired experiments).

In order to validate the method, where the steady-state is disrupted, a comparison using the protocol illustrated in FIG. 3f was used, and as it was expected, their outputs were highly correlated. This shows that the difference in the average metabolic rates obtained by altering the extracellular glucose concentration of 1.2 μM/s compared to the metabolic rate obtained by adding a GLUT inhibitor of 2.0 μM/s is not due to differences between the output of the method but to cell batch variation.

### Temporal Resolution: Acute Activation of Astrocytic Glycolysis by Neuronal Signals

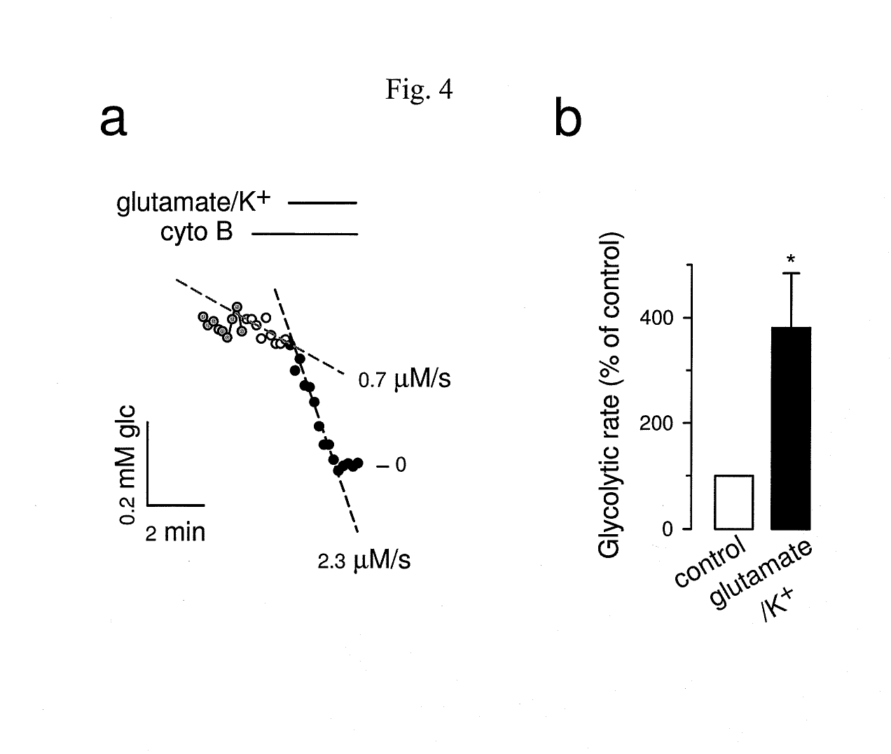

Typical acquisition times in deoxyglucose uptake, deoxyglucose autoradiography and FDG-PET scanning are longer than 10 minutes, but the metabolic changes that characterize the brain tissue develop over seconds. For instance, neuronal activity is accompanied by sub-second shifts in mitochondrial redox potential³, followed in seconds by a rise in interstitial lactate¹¹, which is thought to play key roles in fast neurovascular and neurometabolic coupling¹²⁻¹⁴. Both the identity of the cell type responsible for the lactate surge (i.e. neurons versus astrocytes) and the identity of the local signals that link electrical activity to metabolic activation are controversial^{13,15,16 9}, which is partly explained by the inability of deoxyglucose to monitor events in the range of seconds. Taking advantage of capability of the method of the invention of following glycolytic activation in real-time, cultured astrocytes were exposed simultaneously to glutamate and K⁺, mediators that are co-released by neurons at the excitatory synapse. The data, summarized in FIG. 4, show that the neuronal signals trigger a robust and rapid increase in the rate of glycolysis in astrocytes. Remarkably, the activation was even stronger than the activation achieved by mitochondrial poisoning (FIG. 3e). A control experiment showed that the fast fall in glucose concentration observed in response to glutamate/K⁺ can not be explained by sugar dilution during cell swelling (FIG. 8). By showing for the first time that astrocytic glycolysis can be activated by neuronal signals in the range of seconds, these results suggest that astrocytes are responsible for the lactate surge observed during neuronal activation and lend fresh support to their proposed role in neurometabolic and neurovascular coupling. This level of temporal resolution cannot be obtained with any of the currently available methods or sensors, giving to the present invention a great advantage over them.

### Spatial Resolution: Neurons Versus Astrocytes

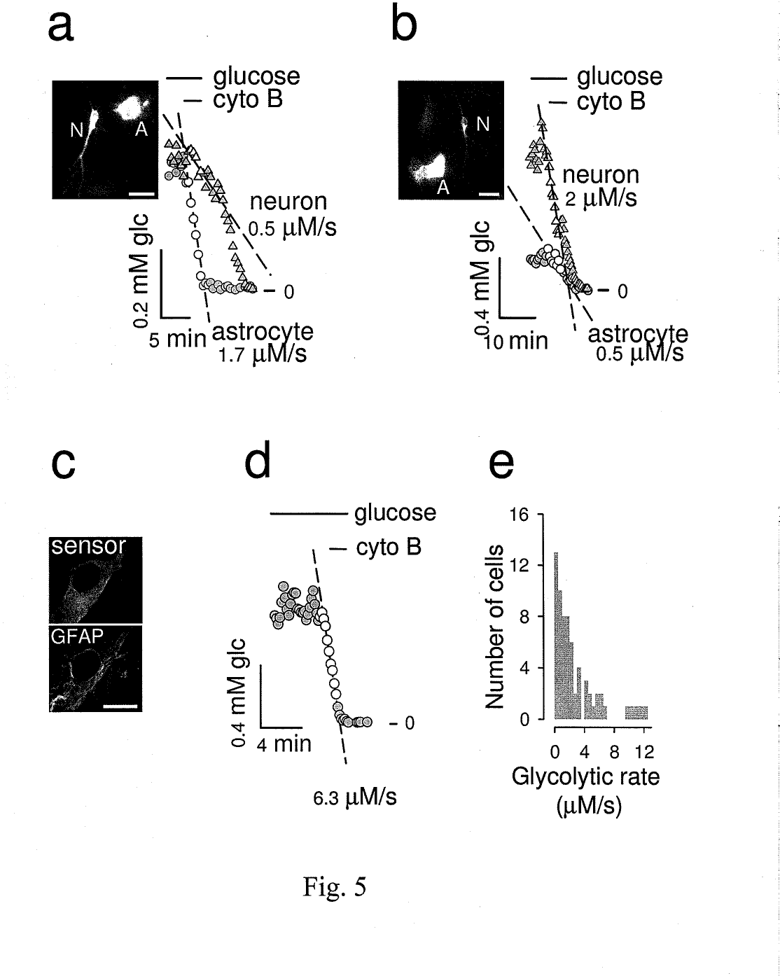

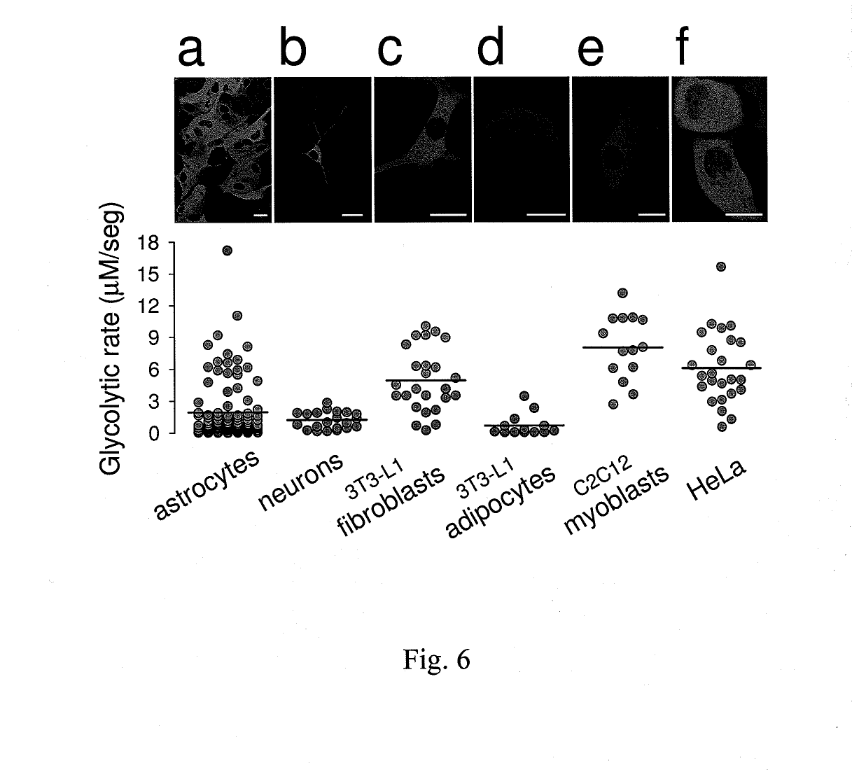

The brain tissue is known in great detail both anatomically and functionally, but its energy usage has not been mapped at high spatial resolution. Each millimeter-sized voxel of an autoradiograph or PET scan is populated by scores of different neuronal and glial types, each cell consuming fuel at a specific rate which is currently not accessible. A way forward is suggested by the two experiments illustrated in FIG. 5a, which shows simultaneous measurement of the glycolytic rate in a neuron and a neighboring astrocyte. We observed great variability of the basal metabolic rate in both cell types but on average the faster cell was the astrocyte (see also FIG. 6). These results in cultured cells do not predict the situation in the brain tissue but given that the FRET nanosensor may be targeted to specific cell types in vivo by means of viral vectors or transgenesis, this kind of measurement may eventually be carried out in the brain tissue in vivo. As an initial step in that direction, the sensor was expressed in hippocampal slices using an adenoviral vector. FIG. 5b shows colocalization between the sensor and the glial protein GFAP, consisting with the preferential targeting of glial cells by these vectors. Similar to that observed in cultured cells, FIG. 5c-d show that the metabolic rate in slices was highly heterogeneous, with an average of 2.8±0.4 μM/s (n=68 cells in twenty five slices, ranging from 0.02 to 12 μM/s).

### Other Cell Types

The metabolic rate could also be measured in other cell types, demonstrating the general applicability of the method (FIG. 6). The results were consistent with previous measurements using radioactive techniques for high rates were observed in undifferentiated and tumor cells (C2C12 myoblasts and HeLa cells) and differentiation of 3T3-L1 fibroblasts into adipocytes led a marked decrease in metabolism. Whereas radioactive techniques and the current methods give similar results, the reliance of the latter on fluorescence should allow their adaptation for the purposes of high-throughput analysis. Moreover, as illustrated in FIG. 6, cultured cell lines showed a high degree of metabolic heterogeneity, perhaps related to cell cycle or other sources of variation, which now seem easier to address.

The working examples described in the present specification are presented with the intention to illustrate the present invention, and are not to be interpreted as a limitation of the scope of the invention.

The present invention introduces new strategies for the measurement of the metabolic rate, whose common rationale is the isolation of glucose phosphorylation by eliminating the contribution of the glucose transporters. The method of the invention offers temporal resolution of seconds and spatial resolution of micrometers, comparing favorably with the method based on 2-deoxyglucose, which offer temporal resolution of minutes and spatial resolution of millimeters. Another important advantage of the present invention is the use of glucose itself, as opposed to glucose analogs, whose handling by hexokinase and posterior fates are usually unknown. One of these analogs, 2-NBDG, the fluorescent version of deoxyglucose, can provide cellular resolution but its rate of uptake is not directly informative about metabolic rate because is orders of magnitude lower than that of glucose and highly sensitive to the properties of the glucose transporter¹⁸⁻²⁰. In previous applications, the FRET glucose nansosensor had been used to measure glucose concentration^{4,6,7}, which depends on both the activity of the glucose transporter and that of hexokinase, and therefore does not inform about metabolic flux. The current approaches circumvent the ambiguity of glucose concentration measurements by providing a direct readout of metabolic flux, without interference from transport properties.

The present invention uses a FRET glucose nanosensor, but any other technique capable of real-time measurement of intracellular glucose concentration may be used for the same purpose. The method of the invention offers cellular resolution and is reversible, presenting different strengths and weaknesses that make them complementary. For example, the method does not require an inhibitor and therefore can be applied to hepatocytes and pancreatic beta-cells, which are rich in GLUT2 and therefore relatively insensitive to cytochalasin B and phloretin. The temporal resolution of the method of the invention is much better and only limited by the time required for data acquisition, usually less the 1 second with high-end setups, providing an extended window of measurement that is ideal for before-and-after kind of experiments such as shown in FIG. 5; however, this method requires a pharmacological inhibitor of GLUT1, which may in principle interfere with metabolism. Although such was not observed to be the case for cytochalasin B in astrocytes, control experiments such as those in FIG. 3 are advised when investigating a new cell type. An improved version of this method would include a yet-to-be-developed potent, specific and ideally non-permeant inhibitor of GLUTs.

In addition to the method of the invention claimed in this application, two original observations were made. Firstly, it was possible for the first time to observe that astrocytic glycolysis can be activated within seconds by neuronal signals. Previous measurements of deoxyglucose uptake over 20 minutes had given conflicting results^{13,15}, but the present method, with its temporal resolution of seconds, was able to show that astrocytic glycolysis is highly sensitive to mediators that are released at the excitatory synapse. The strength and speed of the glycolytic activation observed in astrocytes provides a mechanistic explanation for the fall in glucose concentration and the surge in lactate concentration that can be detected in the brain tissue 5-20 seconds after the onset of neuronal activation^{11, 21-24}, highlighting the role of astrocytes in metabolic coupling and blood flow regulation in the brain^{12,14}. The improved spatial resolution of the method allowed for the first time to monitor the glycolytic rate of a neuron lying on astrocytes. Given the inherent limitations of isotopic measurements, previous work had compared these cells as cultured in isolation, but this is not ideal because both cell types require each other for their proper differentiation and function. Our results in co-culture show high cell-to-cell variability, but on average astrocytes were found to metabolize glucose faster than neurons. Astrocytes could also be studied in brain slices, suggesting that in the near future it will be possible to measure metabolic rates of both cells types in the tissue, in slices and in viva

In order to demonstrate the use of these methods in other cell types, we chose fibroblasts, adipocytes and myoblasts, cells that are widely used for research and drug-screening. The hypoglycemic effect of insulin is explained by an increase in the metabolic rate of adipocytes and muscle cells, a process that becomes defective in diabetes; we envisage that the current methods may be useful for basic and applied research related to this disease. Very high metabolic rates were observed in HeLa, an epithelial cell line of ample use in cancer research. The enhanced glycolytic rate observed in tumor cells, i.e. the Warburg effect, is widely exploited for the purposes of diagnosis and staging by means of FDG-PET scanning, and has recently proposed to play a pathogenic role in cancer progression¹. Thus, the adaptability of the current fluorescent techniques for high-throughput screening and the metabolic heterogeneity evidenced at the single cell level may be instrumental for basic and applied research on cancer.

### REFERENCES

1. Vander Heiden, M. G., Cantley, L. C., & Thompson, C. B. Understanding the Warburg effect: the metabolic requirements of cell proliferation. Science. 324, 1029-1033 (2009).
2. Yeluri, S., Madhok, B., Prasad, K. R., Quirke, P., & Jayne, D. G. Cancer's craving for sugar: an opportunity for clinical exploitation. J. Cancer Res. Clin. Oncol. 135, 867-877 (2009).
3. Brennan, A. M., Connor, J. A., & Shuttleworth, C. W. NAD(P)H fluorescence transients after synaptic activity in brain slices: predominant role of mitochondrial function. J. Cereb. Blood Flow Metab. 26, 1389-1406 (2006).
4. Deuschle, K. et al. Construction and optimization of a family of genetically encoded metabolite sensors by semirational protein engineering. Protein Sci. 14, 2304-2314 (2005).
5. Berg, J., Hung, Y. P., & Yellen, G. A genetically encoded fluorescent reporter of ATP:ADP ratio. Nat. Methods. 6, 161-166 (2009).
6. Fehr, M., Lalonde, S., Lager, I., Wolff, M. W., & Frommer, W. B. In vivo imaging of the dynamics of glucose uptake in the cytosol of COS-7 cells by fluorescent nanosensors. J. Biol. Chem. 278, 19127-19133 (2003).
7. Takanaga, H., Chaudhuri, B., & Frommer, W. B. GLUT1 and GLUT9 as major contributors to glucose influx in HepG2 cells identified by a high sensitivity intramolecular FRET glucose sensor. Biochim. Biophys. Acta. 1778, 1091-1099 (2008).
8. Wilson, J. E. Isozymes of mammalian hexokinase: structure, subcellular localization and metabolic function. J. Exp. Biol. 206, 2049-2057 (2003).
9. Barros, L. F. & Deitmer, J. W. Glucose and lactate supply to the synapse. Brain Res. Rev. (2009).
10. Carruthers, A. Facilitated diffusion of glucose. Physiol Rev. 70, 1135-1176 (1990).
11. Hu, Y. & Wilson, G. S. A temporary local energy pool coupled to neuronal activity: fluctuations of extracellular lactate levels in rat brain monitored with rapid-response enzyme-based sensor. J. Neurochem. 69, 1484-1490 (1997).
12. Magistretti, P. J. Role of glutamate in neuron-glia metabolic coupling. Am. J. Clin. Nutr. 90, 875S-880S (2009).
13. Pellerin, L. et al. Activity-dependent regulation of energy metabolism by astrocytes: an update. Glia. 55, 1251-1262 (2007).
14. Gordon, G. R., Choi, H. B., Rungta, R. L., Ellis-Davies, G. C., & MacVicar, B. A. Brain metabolism dictates the polarity of astrocyte control over arterioles. Nature. 456, 745-749 (2008).
15. Dienel, G. A. & Cruz, N. F. Nutrition during brain activation: does cell-to-cell lactate shuttling contribute significantly to sweet and sour food for thought? Neurochem. Int. 45, 321-351 (2004).
16. Mangia, S., Simpson, I. A., Vannucci, S. J., & Carruthers, A. The in vivo neuron-to-astrocyte lactate shuttle in human brain: evidence from modeling of measured lactate levels during visual stimulation. J. Neurochem. 109 Suppl 1, 55-62 (2009).
17. Hewett, J. A. Determinants of regional and local diversity within the astroglial lineage of the normal central nervous system. J. Neurochem. 110, 1717-1736 (2009).
18. Yamada, K., Saito, M., Matsuoka, H., & Inagaki, N. A real-time method of imaging glucose uptake in single, living mammalian cells. Nat. Protoc. 2, 753-762 (2007).
19. Loaiza, A., Porras, O. H., & Barros, L. F. Glutamate triggers rapid glucose transport stimulation in astrocytes as evidenced by real-time confocal microscopy. J. Neurosci. 23, 7337-7342 (2003).
20. Barros, L. F. et al. Preferential transport and metabolism of glucose in Bergmann glia over Purkinje cells: a multiphoton study of cerebellar slices. Glia 57, 962-970 (2009).
21. Silver, I. A. & Erecinska, M. Extracellular glucose concentration in mammalian brain: continuous monitoring of changes during increased neuronal activity and upon limitation in oxygen supply in normo-, hypo-, and hyperglycemic animals. J. Neurosci. 14, 5068-5076 (1994).
22. Hu, Y. & Wilson, G. S. Rapid changes in local extracellular rat brain glucose observed with an in vivo glucose sensor. J. Neurochem. 68, 1745-1752 (1997).
23. Mangia, S. et al. The aerobic brain: lactate decrease at the onset of neural activity. Neuroscience. 118, 7-10 (2003).
24. Caesar, K. et al. Glutamate receptor-dependent increments in lactate, glucose and oxygen metabolism evoked in rat cerebellum in vivo. J. Physiol. 586, 1337-1349 (2008).
25. Nie, Y. & Wong, C. Suppressing the activity of ERRalpha in 3T3-L1 adipocytes reduces mitochondrial biogenesis but enhances glycolysis and basal glucose uptake. J. Cell Mol. Med. (2008).
26. Ridoux, V., Robert, J., Perricaudet, M., Mallet, J., & Le Gal La, S. G. Adenovirus mediated gene transfer in organotypic brain slices. Neurobiol. Dis. 2, 49-54 (1995).
27. Barros, L. F. Measurement of sugar transport in single living cells. Pflugers Arch. 437, 763-770 (1999).
28. Barros, L. F., Bittner, C. X., Loaiza, A., & Porras, O. H. A quantitative overview of glucose dynamics in the gliovascular unit. Glia. 55, 1222-1237 (2007).

1. A method for the measurement of glucose metabolic rate with high temporal resolution, comprising the steps of:
a. providing single cells or cell populations, cells in suspension or adherent, in a cell culture, a tissue culture, a mixed cell culture, a tissue explant, or in animal tissues in vivo for the measurement of glucose metabolic rate;
b. expressing a glucose sensor in individual cells;
c. calibrating the sensor in controlled conditions;
d. disrupting the steady-state of glucose entering the cell;
e. recording the output from the sensor and calculating the corresponding glucose concentration at different times; and
f. determining the glucose metabolic rate.

2. The method of claim 1, wherein the glucose sensor of step (b) is a fluorescent sensor.

3. The method of claim 2, wherein the glucose sensor is FLIPglu600μΔ11.

4. The method of claim 1, wherein the calibrating of step (c) is performed by pre-treating the cells for 30 minutes with 0.5 mM iodoacetic acid to block glycolysis.

5. The method of claim 4, further comprising:
exposing the cells to increasing concentrations of glucose and monitoring a signal given by the sensor; and
plotting the sensor signal versus glucose concentration to estimate saturation parameters for each different cell type.

6. The method of claim 5, further comprising:
measuring the signal transiently in cells in the absence of glucose to form a zero reading; and
transforming the sensor signal with the zero reading and saturation parameters into glucose concentration.

7. The method of claim 1, wherein the disruption of the glucose steady-state is achieved by lowering extracellular glucose to a concentration lower than the intracellular concentration during the initial steady-state.

8. The method of claim 1, wherein the disruption of the glucose steady-state is achieved by adding a glucose transporter inhibitor to the extracellular space.

9. The method of claim 8, wherein the glucose transporter inhibitor comprises cytochalasin B, phloretin, genistein, parachloromercurybenzoate, or anti-GLUT antisera.

10. The method of claim 8, wherein the glucose transporter inhibitor is cytochalasin B and is added to the extracellular space in a final concentration between 5 μM and 20 μM.

11. The method of claim 1, wherein the output of the glucose sensor is recorded at intervals of time from 100 ms to 1 minute.

12. The method of claim 1, wherein determining the glucose metabolic rate comprises fitting a monoexponential function to the time course of decay and computing the instantaneous slope.

13. A method for the determination of cancer staging, comprising determining the glucose metabolic rate of a sample of cancerous tissue according to the method of claim 1.

14. The method of claim 13, wherein the cancerous tissue is breast cancer, bladder cancer, colon cancer, glioblastoma, lung cancer, hepatocellular carcinoma, gastric cancer, melanoma, thyroid cancer, endometrial cancer, kidney cancer, cervix cancer, pancreatic cancer, esophagus cancer, prostate cancer, brain cancer, ovary cancer, small cell lung cancer, non small cell lung cancer, head and neck cancer, mesothelioma, sarcoma, pediatric malignancies, cholangiocarcinoma.

15. A method for screening potential drugs, comprising determining the glucose metabolic rate of a model cell culture exposed to the potential drug according to the method of claim 1.

16. The method of claim 15, wherein the model cell culture is a culture of 3T3-L1 fibroblast and adipocytes, C2C12 myoblasts and myocytes.

17. The method of claim 15, wherein the model cell culture is a culture of HeLa cells, Neuro 2A, Caco2, C6, A549, MCF7, PC-3, and AGS.
